(19) United States
(12) Patent Application Publication
(10) Pub. No.: US 2022/0033430 A1
(43) Pub. Date: Feb. 3, 2022

(54) ORALLY ACTIVE PRODRUG OF GEMCITABINE

(71) Applicant: TNT Medical Corporation, Zurich (CH)

(72) Inventors: Xiang Li, Pleasanton, CA (US); Bing Ying Li, Pleasanton, CA (US); Starr Sing Chung Cheng, Pleasanton, CA (US); Andrew Wang, Pleasanton, CA (US)

(21) Appl. No.: 17/296,403

(22) PCT Filed: Nov. 22, 2019
(86) PCT No.: PCT/US2019/062747

§ 371 Date: May 24, 2021

Related U.S. Application Data
(60) Provisional application No. 62/771,100, filed on Nov. 25, 2018.

(51) Int. Cl. C07H 19/06; A61P 35/00

(52) U.S. Cl. CPC C07H 19/06; A61P 35/00

(57) ABSTRACT

The disclosure includes compounds of Formula (I): wherein R₁, R₂, and R₃, are defined herein. Also disclosed is a method for treating a neoplastic disease with these compounds.

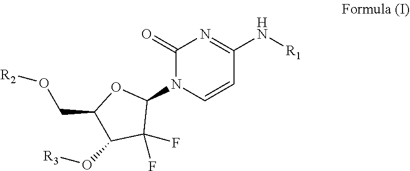

### REFERENCE TO RELATED APPLICATION

This Application claims the benefit of the filing date of U.S. Provisional Patent Application No. 62/771,100, filed on Nov. 25, 2018, the entire content of which is incorporated herein by reference.

### BACKGROUND

Gemcitabine, as shown below, is a pyrimidine nucleoside analogue, shown to be active against several solid tumor types. Following FDA approval in 1996, gemcitabine has become the standard of care for the treatment of pancreatic cancer. More recently, the compound has also gained approval for treating non-small cell lung, ovarian, bladder, and breast cancer.

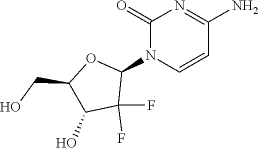 [SMILES: Nc1ccn([C@@H]2O[C@H](CO)[C@@H](O)C2(F)F)c(=O)n1]

### The Chemical Structure of Gemcitabine

Gemcitabine is currently administered by intravenous infusion at a dose of approximately 1000 to 1250 mg/m2 over 30 minutes, once weekly for up to 7 weeks followed by a week of rest from treatment. The use of gemcitabine orally may be limited by its poor oral bioavailability which is the result of first pass metabolism. Shipley L A. Et. al., “Metabolism and disposition of gemcitabine, and oncolytic deoxycytidine analog, in mice, rats, and dogs”. Drug Metabolism & Disposition. 20(6):849-55, 1992. In addition, when dosed orally, gemcitabine is implicated in causing adverse dose-limiting intestinal lesions characterized by moderate-to-marked loss of mucosal epithelium (atrophic enteropathy) throughout the entire length of the intestinal tract in mice given a single oral (gavage) gemcitabine dose of 167, 333, or 500 mg/kg. Horton N D et. al., “Toxicity of single-dose oral gemcitabine in mice”, American Association for Cancer Research, Poster Presentation, Orlando, Fla., Mar. 27-31, 2004. Comparable exposures via intravenous dosing in previous mouse studies did not result in death or gastrointestinal toxicity.

Methods for making orally active prodrug of gemcitabine was reported in the art. In 2009, Bender et al. reported an orally active prodrug of gemcitabine, LY2334737 which is significantly less prone to degradation by CDA due to a valproic acid linkage at the 4-(N)-position. Based on in vivo data in the HCT-116 human colon xenograft, LY2334737 has been further developed and advanced into phase I clinical studies. However, the development was terminated in after unexpected hepatic toxicities were observed with LY2334737 QD in a study of Japanese patients in 2013.

In summary, although LY2334737 have made a significant contribution to the art, there is a continuing search in this field of art for orally active prodrug of gemcitabine.

### SUMMARY OF THE INVENTION

The present invention relates to a novel class of orally active prodrug of Gemciatbine. As shown in the gemcitabine chemical structure above, Gemcitabine has three functional groups (i.e. —OH, —OH, —NH₂) that are amenable to chemical prodrug derivatization. Accordingly, we rationally design an orally active Triple-Prodrug, which all of the three functional groups of Gemcitabine (i.e. —OH, —OH, —NH₂) are simultaneously derivatized with the classic Pro-moieties. (In Prodrug design, Pro-moiety means a chemical functional group used to modify the structure of parent drug to improve physicochemical, biopharmaceutical or pharmacokinetic properties. Pro-moiety is typically biological inactive and safe). Thus, the orally active triple prodrugs of Gemcitabine in the present invention may be useful in treating a patient having a tumor.

This invention provides compounds of the Formula (I), or an N-oxide thereof, or a pharmaceutically acceptable salt, solvate, polymorph, tautomer, stereoisomer, an isotopic form, or a prodrug of said compound of Formula (I) or N-oxide thereof:

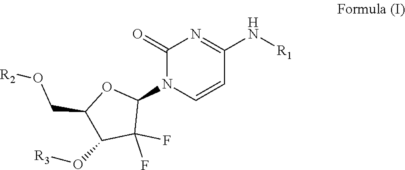

wherein

each of R₁, R₂, and R₃, independently, is

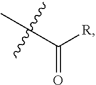

where in R is alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said alkyl, spiroalkyl, alkenyl, alkynyl, cycloalkyl, cycloalkenyl, heterocycloalkyl, spiroheterocycloalkyl, heterocycloalkenyl, aryl, or heteroaryl is optionally substituted with one or more R_{d};

Rₐ, R_{b}, R_{c} and R_{d}, independently, is H, D, alkyl, spiroalkyl, alkenyl, alkynyl, halo, cyano, amine, nitro, hydroxy, ═O, —P(O)R_{b}R_{c}, -alkyl-P(O)R_{b}R_{c}, —S(O)(═N(R_{b}))R_{c}, —N═S(O)R_{b}R_{c}, ═NR_{b}, C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;

Rₑ is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;

In preferred embodiments, the compound is represented by Formula (II):

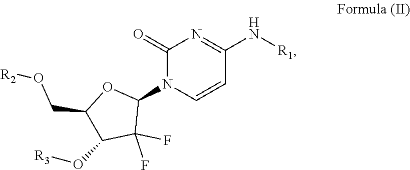

wherein

R₁ is

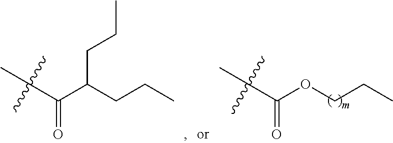

in which m is an integer from 1 to 20; and

each of R₂, and R₃, independently, is

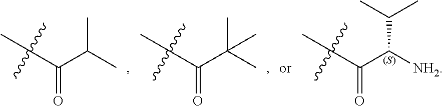

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above for use in treating a neoplastic disease, therapeutic uses thereof, and use of the compounds for the manufacture of a medicament for treating the disease/disorder.

This invention also relates to a method of treating a Pim-overexpressed neoplastic disease, including but not limited to leukemia, lymphoma, multiple myeloma, prostate cancer, pancreatic cancer, gastric cancer, colon cancer, or liver cancer, by administering to a subject in need thereof an effective amount of one or more of the compounds, modifications, and/or salts, and compositions thereof described above.

The details of one or more embodiments of the invention are set forth in the description below. Other features, objects, and advantages of the invention will be apparent from the description and from the claims. It should be understood that all mebodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
(2R,3R,5R)-4,4-difluoro-2-((isobutyryloxy)methyl)-5-(2-oxo-4-(2-propylpentanamido)pyrimidin-1(2H)-yl)tetrahydrofuran-3-yl isobutyrate,
(2R,3R,5R)-4,4-difluoro-2-((isobutyryloxy)methyl)-5-(2-oxo-4-(2-propylpentanamido)pyrimidin-1(2H)-yl)tetrahydrofuran-3-yl L-valinate,
((2R,3R,5R)-3-((L-valyl)oxy)-4,4-difluoro-5-(2-oxo-4-(2-propylpentanamido)pyrimidin-1(2H)-yl)tetrahydrofuran-2-yl)methyl valinate,
((2R,3R,5R)-4,4-difluoro-3-(isobutyryloxy)-5-(2-oxo-4-(2-propylpentanamido)pyrimidin-1(2H)-yl)tetrahydrofuran-2-yl)methyl L-valinate,
(2R,3R,5R)-4,4-difluoro-2-((isobutyryloxy)methyl)-5-(2-oxo-4-(((pentyloxy)carbonyl)amino)pyrimidin-1(2H)-yl)tetrahydrofuran-3-yl L-valinate,
((2R,3R,5R)-4,4-difluoro-3-(isobutyryloxy)-5-(2-oxo-4-(((pentyloxy)carbonyl)amino)pyrimidin-1(2H)-yl)tetrahydrofuran-2-yl)methyl L-valinate,
(2R,3R,5R)-4,4-difluoro-2-((isobutyryloxy)methyl)-5-(2-oxo-4-(((pentyloxy)carbonyl)amino)pyrimidin-1(2H)-yl)tetrahydrofuran-3-yl isobutyrate,
((2R,3R,5R)-3-((L-valyl)oxy)-4,4-difluoro-5-(2-oxo-4-(((pentyloxy)carbonyl)amino)pyrimidin-1(2H)-yl)tetrahydrofuran-2-yl)methyl L-valinate,
(2R,3R,5R)-4,4-difluoro-5-(4-(((hexyloxy)carbonyl)amino)-2-oxopyrimidin-1(2H)-yl)-2-((isobutyryloxy)methyl)tetrahydrofuran-3-yl L-valinate,
(2R,3R,5R)-5-(4-((butoxycarbonyl)amino)-2-oxopyrimidin-1(2H)-yl)-4,4-difluoro-2-((isobutyryloxy)methyl)tetrahydrofuran-3-yl L-valinate,
(2R,3R,5R)-4,4-difluoro-5-(2-oxo-4-(2-propylpentanamido)pyrimidin-1(2H)-yl)-2-((pivaloyloxy)methyl)tetrahydrofuran-3-yl L-valinate,
(2R,3R,5R)-4,4-difluoro-5-(4-(((hexyloxy)carbonyl)amino)-2-oxopyrimidin-1(2H)-yl)-2-((pivaloyloxy)methyl)tetrahydrofuran-3-yl L-valinate.
(2R,3R,5R)-5-(4-(cyclohexanecarboxamido)-2-oxopyrimidin-1(2H)-yl)-4,4-difluoro-2-((isobutyryloxy)methyl)tetrahydrofuran-3-yl L-valinate,
(2R,3R,5R)-5-(4-(cycloheptanecarboxamido)-2-oxopyrimidin-1(2H)-yl)-4,4-difluoro-2-((isobutyryloxy)methyl)tetrahydrofuran-3-yl L-valinate,
(2R,3R,5R)-5-(4-(2,6-dimethyltetrahydro-2H-pyran-4-carboxamido)-2-oxopyrimidin-1(2H)-yl)-4,4-difluoro-2-((isobutyryloxy)methyl)tetrahydrofuran-3-yl L-valinate,
(2R,3R,5R)-4,4-difluoro-2-((isobutyryloxy)methyl)-5-(2-oxo-4-(2-propylhexanamido)pyrimidin-1(2H)-yl)tetrahydrofuran-3-yl L-valinate,
(2R,3R,5R)-5-(4-(2-ethylhexanamido)-2-oxopyrimidin-1(2H)-yl)-4,4-difluoro-2-((isobutyryloxy)methyl)tetrahydrofuran-3-yl L-valinate,
(2R,3R,5R)-4,4-difluoro-2-((isobutyryloxy)methyl)-5-(2-oxo-4-pivalamidopyrimidin-1(2H)-yl)tetrahydrofuran-3-yl L-valinate,
(2R,3R,5R)-5-(4-(4-(tert-butyl)benzamido)-2-oxopyrimidin-1(2H)-yl)-4,4-difluoro-2-((isobutyryloxy)methyl)tetrahydrofuran-3-yl L-valinate,
(2R,3R,5R)-4,4-difluoro-2-((isobutyryloxy)methyl)-5-(4-octanamido-2-oxopyrimidin-1(2H)-yl)tetrahydrofuran-3-yl L-valinate.

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Prodrug derivatives: prodrugs, upon administration to a subject, will converted in vivo into active compounds of the present invention [Nature Reviews of Drug Discovery, 2008, Volume 7, p255]. It is noted that in many instances, the prodrugs themselves also fall within the scope of the range of compounds according to the present invention. The prodrugs of the compounds of the present invention can be prepared by starndard organic reaction, for example, by reacting with a carbamylating agent (e.g., 1,1-acyloxyalkylcarbonochloridate, para-nitrophenyl carbonate, or the like) or an acylating agent. Further examples of methods and strategies of making prodrugs are described in Bioorganic and Medicinal Chemistry Letters, 1994, Vol. 4, p. 1985.
Deuterium-enriched compounds: deuterium (D or 2H) is a stable, non-radioactive isotope of hydrogen and has an atomic weight of 2.0144. Hydrogen naturally occurs as a mixture of the isotopes ^{X} H (hydrogen or protium), D (2H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPPCD and SBE3CD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl. The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

The term “heterocycloalkyl” refers to a nonaromatic 5-8 membered monocyclic, 8-12 membered bicyclic, or 11-14 membered tricyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, or 11-14 membered tricyclic ring system having one or more heteroatoms (such as O, N, S, P, or Se) and one or more double bonds.

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl. The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, aryl, and heteroaryl mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C1-C₁₀ alkoxy, aryl, aryloxy, heteroaryl, heteroaryloxy, amino, C1-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-p-cyclodextrin or sulfobutyl ether O-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDR/VEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDCl₄2BPA, MRCKb/, CDCl₄2BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TAO1, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEEl, WNK1, WNK2, WNK3, YES/YES1, ZAK/MLTK, ZAP70, ZIPK/DAPK3, KINASE, MUTANTS, ABL1(E255K), ABL1(F317I), ABL1(G250E), ABL1(H396P), ABL1(M351T), ABL1(Q252H), ABL1(T315I), ABL1(Y253F), ALK (C1156Y), ALK(L1196M), ALK (F1174L), ALK (R1275Q), BRAF(V599E), BTK(E41K), CHK2(I157T), c-Kit(A829P), c-KIT(D816H), c-KIT(D816V), c-Kit(D820E), c-Kit(N822K), C-Kit (T670I), c-Kit(V559D), c-Kit(V559D/V654A), c-Kit(V559D/T670I), C-Kit (V560G), c-KIT(V654A), C-MET(D1228H), C-MET(D1228N), C-MET(F1200I), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (I2020T), LRRK2 (R1441C), p38a(T106M), PDGFRa(D842V), PDGFRa(T674I), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), antibody conjugate(e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, anti-metabolites, topoisomerase inhibitors, anti-microtubule agents, kinase inhibitors, epigenetic agents, HSP90 inhibitors, PARP inhibitors, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

The invention further provides methods for the prevention or treatment of a neoplastic disease or autoimmune disease. In one embodiment, the invention relates to a method of treating a neoplastic disease or autoimmune disease, in a subject in need of treatment comprising administering to said subject a therapeutically effective amount of a compound of the invention. In one embodiment, the invention further provides for the use of a compound of the invention in the manufacture of a medicament for halting or decreasing a neoplastic disease or autoimmune disease.

In certain embodiments, the neoplastic disease is a lung cancer, head and neck cancer, central nervous system cancer, prostate cancer, testicular cancer, colorectal cancer, pancreatic cancer, liver cancer, stomach cancer, biliary tract cancer, esophageal cancer, gastrointestinal stromal tumor, breast cancer, cervical cancer, ovarian cancer, uterine cancer, leukemia, lymphomas, multiple myeloma, melanoma, basal cell carcinoma, squamous cell carcinoma, bladder cancer, renal cancer, sarcoma, mesothelioma, thymoma, myelodysplastic syndrome, or myeloproliferative disease.

The autoimmune diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

A typical approach to synthesize of Formula (1) compounds is described in Scheme A. R₁, R₂, and R₃, in general Scheme A are the same as those described in the Summary section above.

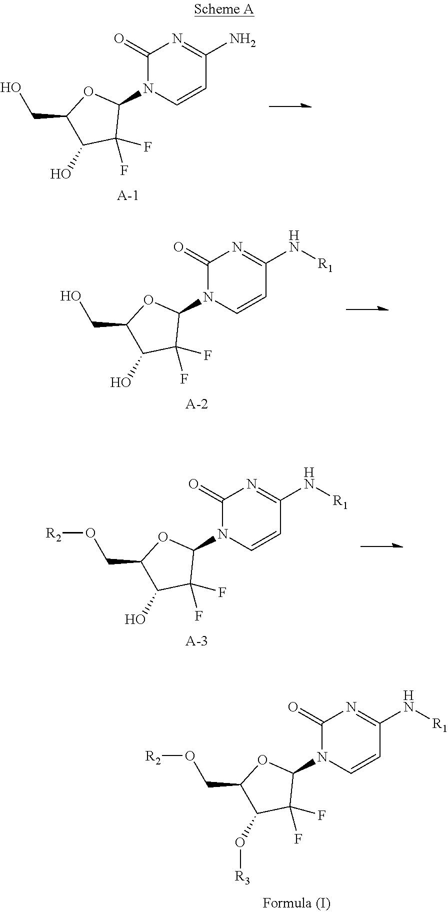

In Scheme A, the starting material Gemcitabine can react with 2-propylpentanoic acid or appropriate alkyl carbonochloridate to yield intermediate A-2, which can react with appropriate acyl chloride or carboxylic acid to form the intermediate A-3. Finally, A-3 can react with appropriate acyl chloride or carboxylic acid to form the desired final product with Formula (I).

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example 1: Synthesis of [(2R,3R,5R)-4,4-difluoro-3-[(2-methylpropanoyl)oxy]-5-[2-oxo-4-(2-propylpentanamido)-1,2-dihydropyrimidin-1-yl]oxolan-2-yl]methyl 2-methylpropanoate

Into a 500-mL 3-necked round-bottom flask purged and maintained with an inert atmosphere of nitrogen was placed a solution of 2-propylpentanoic acid (12 g, 83.21 mmol, 1.30 equiv), HOBt (10.27 g, 76.01 mmol, 1.15 equiv), NMM (7.67 g, 75.83 mmol, 1.15 equiv) and EDCI.HCl (18.87 g, 1.30 equiv) in N,N-dimethylformamide (60 mL). To above solution 4-amino-1-[(2R,4R,5R)-3,3-difluoro-4-hydroxy-5-(hydroxymethyl)oxolan-2-yl]-1,2-dihydropyrimidin-2-one hydrochloride (20 g, 66.74 mmol, 1.00 equiv) in DMF (20 mL) was added at RT. The resulting solution was stirred overnight at 55° C. in an oil bath. The reaction was then quenched by the addition of 200 mL of brine. The resulting solution was extracted with 3×50 mL of ethyl acetate and the organic layers combined. The resulting mixture was washed with 1×50 mL of aqueous HCl and 1×50 mL of brine. The resulting mixture was dried and concentrated under vacuum. The residue was applied onto a silica gel column with ethyl acetate/petroleum ether (1:3). This resulted in 17.5 g (67%) of N-[1-[(2R,4R,5R)-3,3-difluoro-4-hydroxy-5-(hydroxymethyl)oxolan-2-yl]-2-oxo-1,2-dihydropyrimidin-4-yl]-2-propylpentanamide as a off-white solid. (ES, m/z): [M+H]⁺=390. ¹H-NMR: (300 MHz, CDCl₃, ppm): δ 8.80 (br, 1H), 8.21 (d, J=7.8 Hz, 1H), 7.57 (d, J=7.8 Hz, 1H), 6.26 (t, J=6.7 Hz, 1H), 5.20 (br, 1H), 4.53 (m, 1H), 4.15-3.90 (m, 4H), 2.39 (br, 1H), 1.69-1.21 (m, 8H), 0.92 (t, J=7.2 Hz, 6H).

Into a 25-mL round-bottom flask purged and maintained with an inert atmosphere of nitrogen, was placed N-[1-[(2R,4R,5R)-3,3-difluoro-4-hydroxy-5-(hydroxymethyl)oxolan-2-yl]-2-oxo-1,2-dihydropyrimidin-4-yl]-2-propylpentanamide (500 mg, 1.28 mmol, 1.00 equiv), 2-methylpropanoyl chloride (272 mg, 2.55 mmol, 2.20 equiv), 4-dimethylaminopyridine (16 mg, 0.13 mmol, 0.10 equiv). This was followed by the addition of pyridine (5 mL) at 0° C. and the resulting solution was stirred overnight at room temperature. The resulting mixture was concentrated under vacuum and purified by Flash-Prep-HPLC. This resulted in 167 mg (24%) of [(2R,3R,5R)-4,4-difluoro-3-[(2-methylpropanoyl)oxy]-5-[2-oxo-4-(2-propylpentanamido)-1,2-dihydropyrimidin-1-yl]oxolan-2-yl]methyl 2-methylpropanoate as light brown semi-solid. LC-MS: (ES, m/z): [M+H]⁺=530. ¹H-NMR: (300 MHz, d₆-DMSO, ppm): δ 11.11 (s, 1H), 8.06 (d, J=7.8 Hz, 1H), 7.38 (d, J=7.8 Hz, 1H), 6.33 (t, J=8.7 Hz, H), 5.45 (q, J=6.0 Hz, 1H), 4.52-4.36 (m, 3H), 2.76-2.52 (m, 3H), 1.61-1.03 (m, 20H), 0.86 (t, J=7.2 Hz, 3H).

### Example 2: Synthesis of (2R,3R,5R)-4,4-difluoro-2-[[(2-methylpropanoyl)oxy]methyl]-5-[2-oxo-4-(2-propylpentanamido)-1,2-dihydropyrimidin-1-yl]oxolan-3-yl (2S)-2-amino-3-methylbutanoate

Into a 50-mL round-bottom flask, was placed 2-methylpropanoic acid (170 mg, 1.93 mmol, 1.50 equiv), CDI (0.31 g, 1.93 mmol, 1.50 equiv), tetrahydrofuran (30 mL). This was followed by the addition of N-[1-[(2R,4R,5R)-3,3-difluoro-4-hydroxy-5-(hydroxymethyl)oxolan-2-yl]-2-oxo-1,2-dihydropyrimidin-4-yl]-2-propylpentanamide (0.5 g, 1.29 mmol, 1.00 equiv). The resulting solution was stirred for 2 h at room temperature. The resulting mixture was concentrated under vacuum. The crude product was purified by Flash, PE:EA=100/20 increasing to PE:EA=100/50 within 20 min. This resulted in 0.45 g (76%) of [(2R,3R,5R)-4,4-difluoro-3-hydroxy-5-[2-oxo-4-(2-propylpentanamido)-1,2-dihydropyrimidin-1-yl]oxolan-2-yl]methyl 2-methylpropanoate as white oil. ¹H-NMR: (300 MHz, d₆-DMSO, ppm): δ 11.12 (s, 1H), 8.21 (d, J=7.8 Hz, 1H), 7.35 (d, J=7.8 Hz, 1H), 6.30 (t, J=8.7 Hz, 1H), 5.47-5.30 (m, 2H), 4.27 (m, 1H), 3.84-3.58 (m, 2H), 2.66-2.55 (m, 2H), 1.60-1.10 (m, 14H), 0.88 (t, J=7.1 Hz, 6H).

Into a 50-mL round-bottom flask, was placed [(2R,3R,5R)-4,4-difluoro-3-hydroxy-5-[2-oxo-4-(2-propylpentanamido)-1,2-dihydropyrimidin-1-yl]oxolan-2-yl]methyl 2-methylpropanoate (0.4 g, 0.87 mmol, 1.00 equiv), (2S)-2-[[(tert-butoxy)carbonyl]amino]-3-methylbutanoic acid (380 mg, 4.36 mmol, 2.00 equiv), DCC (360 mg, 4.37 mmol, 2.00 equiv), 4-dimethylaminopyridine (215 mg, 4.34 mmol, 2.00 equiv), N,N-dimethylformamide (15 mL). The resulting solution was stirred for 2 h at room temperature. The reaction was then quenched by the addition of H₂O. The resulting solution was extracted with of ethyl acetate and the organic layers combined and concentrated under vacuum. The crude product was purified by Flash PE:EA=100/20 increasing to PE:EA=100/60 within 30 min. This resulted in 0.52 g (91%) of (2R,3R,5R)-4,4-difluoro-2-[[(2-methylpropanoyl)oxy]methyl]-5-[2-oxo-4-(2-propylpentanamido)-1,2-dihydropyrimidin-1-yl]oxolan-3-yl (2S)-2-[[(tert-butoxy)carbonyl]amino]-3-methylbutanoate as white oil.

Into a 50-mL round-bottom flask, was placed (2R,3R,5R)-4,4-difluoro-2-[[(2-methylpropanoyl)oxy]methyl]-5-[2-oxo-4-(2-propylpentanamido)-1,2-dihydropyrimidin-1-yl]oxolan-3-yl (2S)-2-[[(tert-butoxy)carbonyl]amino]-3-methylbutanoate (500 mg, 0.76 mmol, 1.00 equiv), hydrogen chloride/Dioxane (4M, 30 mL). The resulting solution was stirred for 1 h at room temperature. The resulting mixture was concentrated under vacuum. The crude product was purified by Prep-HPLC. This resulted in 312 mg (46%) of (2R,3R,5R)-4,4-difluoro-2-[[(2-methylpropanoyl)oxy]methyl]-5-[2-oxo-4-(2-propylpentanamido)-1,2-dihydropyrimidin-1-yl]oxolan-3-yl (2S)-2-amino-3-methylbutanoate as a off-white solid. LC-MS: (M+H)⁺=559. ¹H-NMR: (300 MHz, d₆-DMSO, ppm): δ 11.12 (s, 1H), 8.07 (d, J=7.8 Hz, 1H), 7.38 (d, J=7.8 Hz, 1H), 6.35 (t, J=8.7 Hz, 1H), 5.47 (q, J=6.0 Hz, 1H), 4.55-4.51 (m, 3H), 3.86 (d, J=6.0 Hz, 1H), 2.76-2.71 (m, 2H), 2.15 (m, 1H), 1.59-1.03 (m, 14H), 1.01-0.83 (m, 12H).

### Example 3: Synthesis of [(2R,3R,5R)-4,4-difluoro-3-[(2-methylpropanoyl)oxy]-5-[2-oxo-4-(2-propylpentanamido)-1,2-dihydropyrimidin-1-yl]oxolan-2-yl]methyl (2S)-2-amino-3-methylbutanoate

Into a 100-mL round-bottom flask, was placed (2S)-2-[[(tert-butoxy)carbonyl]amino]-3-methylbutanoic acid (0.4 g, 1.84 mmol, 1.20 equiv), CDI (300 mg, 1.85 mmol, 1.20 equiv), tetrahydrofuran (25 mL). The resulting mixture was stirred 30 min at r.t. To this was added N-[l-[(2R,4R,5R)-3,3-difluoro-4-hydroxy-5-(hydroxymethyl)oxolan-2-yl]-2-oxo-1,2-dihydropyrimidin-4-yl]-2-propylpentanamide (0.6 g, 1.54 mmol, 1.00 equiv) and the resulting solution was stirred overnight at room temperature. The resulting mixture was concentrated under vacuum. The crude product was purified by Flash PE:EA=100/50. This resulted in 0.72 g (79%) of [(2R,3R,5R)-4,4-difluoro-3-hydroxy-5-[2-oxo-4-(2-propylpentanamido)-1,2-dihydropyrimidin-1-yl]oxolan-2-yl]methyl (2R)-2-[[(tert-butoxy)carbonyl]amino]-3-methylbutanoate as white oil. LC-MS: (ES, m/z): 589[M+H]⁺.

Into a 50-mL round-bottom flask, was placed [(2R,3R,5R)-4,4-difluoro-3-hydroxy-5-[2-oxo-4-(2-propylpentanamido)-1,2-dihydropyrimidin-1-yl]oxolan-2-yl]methyl (2R)-2-[[(tert-butoxy)carbonyl]amino]-3-methylbutanoate (700 mg, 1.19 mmol, 1.00 equiv), 4-dimethylaminopyridine (290 mg, 2.38 mmol, 2.00 equiv), 2-methylpropanoyl chloride (153 mg, 1.40 mmol, 1.20 equiv), pyridine (14 mL). The resulting solution was stirred for 1 h at room temperature. The resulting mixture was concentrated under vacuum. The crude product was purified by Prep-HPLC. This resulted in 210 mg (27%) of [(2R,3R,5R)-4,4-difluoro-3-[(2-methylpropanoyl)oxy]-5-[2-oxo-4-(2-propylpentanamido)-1,2-dihydropyrimidin-1-yl]oxolan-2-yl]methyl (2R)-2-[[(tert-butoxy)carbonyl]amino]-3-methylbutanoate as a white solid. LC-MS: (ES, m/z): [M+H]⁺=659. ¹H-NMR: (300 MHz, d₆-DMSO, ppm): δ 11.11 (s, 1H), 8.09 (d, J=7.8 Hz, 1H), 7.38 (d, J=7.8 Hz, 1H), 6.35 (t, J=8.7 Hz, 1H), 5.50 (br, 1H), 4.45-4.28 (m, 2H), 3.93 (m, 1H), 2.72-2.58 (m, 2H), 2.05 (m, 1H), 1.60-1.06 (m, 24H), 0.95-0.84 (m, 12H).

Into a 50-mL round-bottom flask purged and maintained with an inert atmosphere of nitrogen, was placed [(2R,3R,5R)-4,4-difluoro-3-[(2-methylpropanoyl)oxy]-5-[2-oxo-4-(2-propylpentanamido)-1,2-dihydropyrimidin-1-yl]oxolan-2-yl]methyl (2R)-2-[[(tert-butoxy)carbonyl]amino]butanoate (200 mg, 0.32 mmol, 1.00 equiv), hydrogen chloride/Dioxane (2 mL). The resulting solution was stirred for 30 min at room temperature. The resulting mixture was concentrated under vacuum. The residue was purified by pre-HPLC then applied onto a silica gel column with ethyl acetate. This resulted in 35.1 mg (41%) of [(2R,3R,5R)-4,4-difluoro-3-[(2-methylpropanoyl)oxy]-5-[2-oxo-4-(2-propylpentanamido)-1,2-dihydropyrimidin-1-yl]oxolan-2-yl]methyl (2S)-2-amino-3-methylbutanoate as colorless oil. LC-MS: (ES, m/z): [M+H]⁺=559. ¹H-NMR: (300 MHz, d₆-DMSO, ppm): δ 11.11 (s, 1H), 8.07 (d, J=7.8 Hz, 1H), 7.38 (d, J=7.8 Hz, 1H), 6.35 (t, J=8.7 Hz, 1H), 5.50 (q, J=6.0 Hz, 1H), 4.52-4.37 (m, 3H), 3.28 (d, J=6.0 Hz, 1H) 2.67-2.60 (m, 3H), 1.99-1.87 (m, 2H), 1.60-1.06 (m, 15H), 0.95-0.84 (m, 12H).

### Example 4: Synthesis of [(2R,3R,5R)-3-[[(2S)-2-amino-3-methylbutanoyl]oxy]-4,4-difluoro-5-[2-oxo-4-(2-propylpentanamido)-1,2-dihydropyrimidin-1-yl]oxolan-2-yl]methyl (2S)-2-amino-3-methylbutanoate

Into a 50-mL round-bottom flask purged and maintained with an inert atmosphere of nitrogen, was placed a solution of N-[l-[(2R,4R,5R)-3,3-difluoro-4-hydroxy-5-(hydroxymethyl)oxolan-2-yl]-2-oxo-1,2-dihydropyrimidin-4-yl]-2-propylpentanamide (778 mg, 2.00 mmol, 1.00 equiv) in N,N-dimethylformamide (20 mL) and then to the solution was added (2R)-2-[[(tert-butoxy)carbonyl]amino]-3-methylbutanoic acid (1.73 g, 7.96 mmol, 4.00 equiv), 4-dimethylaminopyridine (730 mg, 5.98 mmol, 3.00 equiv), DCC (2.5 g, 12.12 mmol, 6.00 equiv). The resulting solution was stirred for 3 h at room temperature. The reaction was then quenched by the addition of 50 mL of water. The resulting solution was extracted with 3×100 mL of ethyl acetate and the organic layers combined and concentrated under vacuum. The residue was applied onto a silica gel column with ethyl acetate/petroleum ether (1:1). This resulted in 700 mg (44%) of [(2R,3R,5R)-3-[[(2S)-2-[[(tert-butoxy)carbonyl]amino]-3-methylbutanoyl]oxy]-4,4-difluoro-5-[2-oxo-4-(2-propylpentanamido)-1,2-dihydropyrimidin-1-yl]oxolan-2-yl]methyl (2R)-2-[[(tert-butoxy)carbonyl]amino]-3-methylbutanoate as an off-white solid.

Into a 25-mL round-bottom flask, was placed [(2R,3R,5R)-3-[[(2S)-2-[[(tert-butoxy)carbonyl]amino]-3-methylbutanoyl]oxy]-4,4-difluoro-5-[2-oxo-4-(2-propylpentanamido)-1,2-dihydropyrimidin-1-yl]oxolan-2-yl]methyl (2R)-2-[[(tert-butoxy)carbonyl]amino]-3-methylbutanoate (200 mg, 0.13 mmol, 1.00 equiv), hydrogen chloride/Dioxane (4M, 5 mL). The resulting solution was stirred for 1 h at room temperature. The resulting mixture was concentrated under vacuum and recrystallized with MeCN. This resulted in 90 mg (55%) of [(2R,3R,5R)-3-[[(2S)-2-amino-3-methylbutanoyl]oxy]-4,4-difluoro-5-[2-oxo-4-(2-propylpentanamido)-1,2-dihydropyrimidin-1-yl]oxolan-2-yl]methyl (2S)-2-amino-3-methylbutanoate as white solid. LC-MS: (M+H)⁺=588. ¹H-NMR: (300 MHz, d₆-DMSO, ppm): δ 11.13 (s, 1H), 8.12 (d, J=7.8 Hz, 1H), 7.39 (d, J=7.8 Hz, 1H), 6.35 (t, J=8.7 Hz, 1H), 5.65 (m, 1H), 4.68-4.61 (m, 3H), 4.03-3.94 (m, 2H), 2.69-2.60 (m, 1H), 2.30-2.19 (m, 2H), 1.60-1.10 (m, 8H), 1.10-0.95 (m, 12H), 0.84 (t, J=7.1 Hz, 6H).

### Example 5: Synthesis of [(2R,3R,5R)-4,4-difluoro-3-[(2-methylpropanoyl)oxy]-5-(2-oxo-4-[[(pentyloxy)carbonyl]amino]-1,2-dihydropyrimidin-1-yl)oxolan-2-yl]methyl 2-methylpropanoate

Into a 25-mL round-bottom flask purged and maintained with an inert atmosphere of nitrogen, was placed a solution of 4-amino-1-[(2R,4R,5R)-3,3-difluoro-4-hydroxy-5-(hydroxymethyl)oxolan-2-yl]-1,2-dihydropyrimidin-2-one (200 mg, 0.66 mmol, 1.00 equiv) in CH₃CN (2 mL), pentyl chloroformate (126 mg, 0.84 mmol, 1.30 equiv), NMM (153.6 mg, 1.52 mmol, 2.40 equiv). The resulting solution was stirred for 2 h at room temperature. The resulting solution was diluted with 10 mL of water. The resulting solution was extracted with 2×10 mL of ethyl acetate and the organic layers combined and concentrated under vacuum. The residue was applied onto a silica gel column with ethyl acetate/petroleum ether (1:1). This resulted in 90 mg (31%) of pentyl N-[1-[(2R,4R,5R)-3,3-difluoro-4-hydroxy-5-(hydroxymethyl)oxolan-2-yl]-2-oxo-1,2-dihydropyrimidin-4-yl]carbamate as a off-white solid. LC-MS: (ES, m/z): [M+H]⁺=378. ¹H-NMR: (300 MHz, d₆-DMSO, ppm): δ 10.81 (br, 1H), 8.23 (d, J=7.8 Hz, 1H), 7.11 (d, J=7.8 Hz, 1H), 6.31 (d, J=6.3 Hz, 1H), 6.17 (t, J=7.5 Hz, 1H), 5.30 (t, J=5.5 Hz, 1H), 4.20 (m, 1H), 3.93-3.60 (m, 3H), 1.69-1.23 (m, 8H), 0.90 (m, 3H).

Into a 25-mL round-bottom flask purged and maintained with an inert atmosphere of nitrogen, was placed a solution of pentyl-N-[1-[(2R,4R,5R)-3,3-difluoro-4-hydroxy-5-(hydroxymethyl)oxolan-2-yl]-2-oxo-1,2-dihydropyrimidin-4-yl]carbamate (110 mg, 0.29 mmol, 1.00 equiv) in pyridine (1 mL) and then to the solution was added 4-dimethylaminopyridine (10 mg, 0.08 mmol, 0.10 equiv), 2-methylpropanoyl chloride (69 mg, 0.65 mmol, 2.20 equiv). The resulting solution was stirred for 3 h at room temperature. The resulting mixture was concentrated under vacuum. The crude product was purified by Flash-Prep-HPLC and result in 90 mg (60%) of [(2R,3R,5R)-4,4-difluoro-3-[(2-methylpropanoyl)oxy]-5-(2-oxo-4-[[(pentyloxy)carbonyl]amino]-1,2-dihydropyrimidin-1-yl)oxolan-2-yl]methyl 2-methylpropanoate as colorless oil. LC-MS: (ES, m/z): [M+H]⁺=518. ¹H-NMR: (300 MHz, d₆-DMSO, ppm): δ 10.89 (br, 1H), 8.06 (d, J=7.8 Hz, 1H), 7.15 (d, J=7.8 Hz, 1H), 6.35 (t, J=8.7 Hz, 1H), 5.45 (m, 1H), 4.49-4.37 (m, 3H), 4.12 (t, J=6.6 Hz, 2H), 2.77-2.54 (m, 2H), 1.66-1.58 (m, 2H), 1.40-1.04 (m, 16H), 0.90 (m, 3H).

### Example 6: Synthesis of (2R,3R,5R)-5-[4-[(butoxycarbonyl)amino]-2-oxo-1,2-dihydropyrimidin-1-yl]-4,4-difluoro-2-[[(2-methylpropanoyl)oxy]methyl]oxolan-3-yl (2S)-2-amino-3-methylbutanoate

Into a 25-mL round-bottom flask purged and maintained with an inert atmosphere of nitrogen, was placed a solution of 2-methylpropanoic acid (350 mg, 3.97 mmol, 1.50 equiv) in tetrahydrofuran (10 mL). To the solution was added CDI (680 mg, 4.19 mmol, 1.60 equiv) and then the mixture was stirred at r.t. for 30 mins. To the above solution was added pentyl-N-[1-[(2R,4R,5R)-3,3-difluoro-4-hydroxy-5-(hydroxymethyl)oxolan-2-yl]-2-oxo-1,2-dihydropyrimidin-4-yl]carbamate (1.0 g, 2.65 mmol, 1.00 equiv). The resulting solution was stirred for 3 h at room temperature. The resulting mixture was concentrated under vacuum. The residue was applied onto a silica gel column with ethyl acetate/petroleum ether (1:1). This resulted in 500 mg (42%) of [(2R,3R,5R)-4,4-difluoro-3-hydroxy-5-(2-oxo-4-[[(pentyloxy)carbonyl]amino]-1,2-dihydropyrimidin-1-yl)oxolan-2-yl]methyl 2-methylpropanoate as a white solid. LC-MS: (ES, m/z): [M+H]⁺=448. ¹H-NMR: (300 MHz, d₆-DMSO, ppm): δ 10.85 (br, 1H), 8.18 (d, J=7.8 Hz, 1H), 7.14 (d, J=7.8 Hz, 1H), 6.30 (t, J=8.7 Hz, 1H), 5.45-5.30 (m, 2H), 4.25 (m, 1H), 3.85-3.60 (m, 2H), 2.77-2.63 (m, 1H), 1.70-1.58 (br, 2H), 1.40-1.12 (m, 12H), 0.86 (m, 3H).

Into a 25-mL round-bottom flask purged and maintained with an inert atmosphere of nitrogen, was placed a solution of [(2R,3R,5R)-4,4-difluoro-3-hydroxy-5-(2-oxo-4-[[(pentyloxy)carbonyl]amino]-1,2-dihydropyrimidin-1-yl)oxolan-2-yl]methyl 2-methylpropanoate (250 mg, 0.56 mmol, 1.00 equiv) in N,N-dimethylformamide (3 mL), (2S)-2-[[(tert-butoxy)carbonyl]amino]-3-methylbutanoic acid (157.7 mg, 0.73 mmol, 1.30 equiv), DCC (150 mg, 0.73 mmol, 1.30 equiv), 4-dimethylaminopyridine (136 mg, 1.11 mmol, 2.00 equiv). The resulting solution was stirred for 1.5 h at room temperature. The solids were filtered out. The residue was concentrated and applied onto a silica gel column with ethyl acetate/petroleum ether (1:1). This resulted in 320 mg (91%) of (2R,3R,5R)-5-[4-[(butoxycarbonyl)amino]-2-oxo-1,2-dihydropyrimidin-1-yl]-4,4-difluoro-2-[[(2-methylpropanoyl)oxy]methyl]oxolan-3-yl (2S)-2-[[(tert-butoxy)carbonyl]amino]-3-methylbutanoate as a white solid. LC-MS: (ES, m/z): [M+H]⁺=647.

Into a 25-mL round-bottom flask purged and maintained with an inert atmosphere of nitrogen, was placed a solution of (2R,3R,5R)-5-[4-[(butoxycarbonyl)amino]-2-oxo-1,2-dihydropyrimidin-1-yl]-4,4-difluoro-2-[[(2-methylpropanoyl)oxy]methyl]oxolan-3-yl-(2S)-2-[[(tert-butoxy)carbonyl]amino]-3-methylbutanoate (100 mg, 0.16 mmol, 1.00 equiv) in dioxane (2 mL), hydrogen chloride/dioxane (1 mL). The resulting solution was stirred for 1 h at room temperature. The resulting mixture was concentrated under vacuum and purified by pre-HPLC(TFA). This resulted in 50 mg (50%) of (2R,3R,5R)-5-[4-[(butoxycarbonyl)amino]-2-oxo-1,2-dihydropyrimidin-1-yl]-4,4-difluoro-2-[[(2-methylpropanoyl)oxy]methyl]oxolan-3-yl (2S)-2-amino-3-methylbutanoate as a off-white solid. LC-MS: (ES, m/z): [M+H]⁺=547. ¹H-NMR: (300 MHz, d₆-DMSO, ppm): δ 10.92 (br, 1H), 8.40 (br, 3H), 8.05 (d, J=7.8 Hz, 1H), 7.16 (d, J=7.8 Hz, 1H), 6.35 (t, J=8.7 Hz, 1H), 5.50 (m, 1H), 4.62-4.51 (m, 3H), 4.13 (t, J=6.6 Hz, 2H), 4.03 (s, 1H), 2.77-2.67 (m, 1H), 2.21-2.16 (m, 1H) 1.62 (m, 1H), 1.34-1.30 (m, 4H), 1.16 (t, J=5.6 Hz, 1H), 1.05-0.86 (m, 9H).

### Example 7: Synthesis of [(2R,3R,5R)-4,4-difluoro-3-[(2-methylpropanoyl)oxy]-5-(2-oxo-4-[[(pentyloxy)carbonyl]amino]-1,2-dihydropyrimidin-1-yl)oxolan-2-yl]methyl (2R)-2-amino-3-methylbutanoate

Into a 100-mL round-bottom flask, was placed (2S)-2-[[(tert-butoxy)carbonyl]amino]-3-methylbutanoic acid (315 mg, 1.45 mmol, 1.10 equiv), CDI (235 mg, 1.45 mmol, 1.10 equiv). This was followed by the addition of tetrahydrofuran (20 mL) and stirred for 30 min at r.t. To this was added pentyl-N-[l-[(2R,4R,5R)-3,3-difluoro-4-hydroxy-5-(hydroxymethyl)oxolan-2-yl]-2-oxo-1,2-dihydropyrimidin-4-yl]carbamate (0.5 g, 1.32 mmol, 1.00 equiv) dropwise with stirring. The resulting solution was stirred overnight at room temperature. The resulting mixture was concentrated under vacuum. The crude product was purified by Flash-Prep-HPLC PE:EA=95:5 increasing to PE:EA=70:30 within 30 min. This resulted in 0.6 g (79%) of [(2R,3R,5R)-4,4-difluoro-3-hydroxy-5-(2-oxo-4-[[(pentyloxy)carbonyl]amino]-1,2-dihydropyrimidin-1-yl)oxolan-2-yl]methyl (2R)-2-[[(tert-butoxy)carbonyl]amino]-3-methylbutanoate as an off-white solid. ¹H-NMR: (300 MHz, d₆-DMSO, ppm): δ 10.86 (br, 1H), 8.20 (d, J=7.8 Hz, 1H), 7.40 (d, J=7.8 Hz, 1H), 7.13 (d, J=7.5 Hz, 1H), 6.30 (t, J=8.7 Hz, 1H), 5.50-5.30 (m, 2H), 4.22 (m, 1H), 4.10 (t, J=6.3 Hz, 2H), 3.97-59(m, 3H), 2.05 (m, 1H), 1.66 (m, 2H), 1.35-1.25 (m, 13H), 0.91 (m, 9H).

Into a 50-mL round-bottom flask, was placed [(2R,3R,5R)-4,4-difluoro-3-hydroxy-5-(2-oxo-4-[[(pentyloxy)carbonyl]amino]-1,2-dihydropyrimidin-1-yl)oxolan-2-yl]methyl 2-[[(tert-butoxy)carbonyl]amino]-3-methylbutanoate (0.5 g, 0.86 mmol, 1.00 equiv), 2-methylpropanoyl chloride (110 mg, 1.03 mmol, 1.20 equiv), 4-dimethylaminopyridine (212 mg, 1.74 mmol, 2.00 equiv), pyridine (10 mL). The resulting solution was stirred for 2 h at room temperature. The resulting mixture was concentrated under vacuum. The resulting solution was extracted with of methanol and the organic layers combined. The crude product was purified by Prep-HPLC. This resulted in 300 mg (53%) of [(2R,3R,5R)-4,4-difluoro-3-[(2-methylpropanoyl)oxy]-5-(2-oxo-4-[[(pentyloxy)carbonyl]amino]-1,2-dihydropyrimidin-1-yl)oxolan-2-yl]methyl 2-[[(tert-butoxy)carbonyl]amino]-3-methylbutanoate as a white solid. ¹H-NMR: (300 MHz, d₆-DMSO, ppm): δ 10.89 (br, 1H), 8.06 (d, J=7.8 Hz, 1H), 7.42 (d, J=7.8 Hz, 1H), 7.15 (d, J=7.5 Hz, 1H), 6.35 (t, J=8.7 Hz, 1H), 5.55 (m, 1H), 4.55-4.30 (m, 3H), 4.10 (t, J=6.3 Hz, 2H), 3.92 (m, 1H), 2.70-2.59 (m, 1H), 2.10 (m, 1H), 1.68-1.55 (m, 2H), 1.45-1.27 (m, 13H), 1.19-1.05 (m, 6H), 0.91 (m, 9H).

Into a 25-mL round-bottom flask, was placed [(2R,3R,5R)-4,4-difluoro-3-[(2-methylpropanoyl)oxy]-5-(2-oxo-4-[[(pentyloxy)carbonyl]amino]-1,2-dihydropyrimidin-1-yl)oxolan-2-yl]methyl (2S)-2-[[(tert-butoxy)carbonyl]amino]-3-methylbutanoate (100 mg, 0.08 mmol, 1.00 equiv), hydrogen chloride/Dioxane (5 mL). The resulting solution was stirred for 30 min at room temperature. The resulting mixture was concentrated under vacuum. The crude product was purified by Prep-HPLC. This resulted in 35.8m g (35%) of [(2R,3R,5R)-4,4-difluoro-3-[(2-methylpropanoyl)oxy]-5-(2-oxo-4-[[(pentyloxy)carbonyl]amino]-1,2-dihydropyrimidin-1-yl)oxolan-2-yl]methyl (2R)-2-amino-3-methylbutanoate as a white solid. LC-MS: (M+H)⁺=547. ¹H-NMR: (300 MHz, CD₃OD, ppm): δ 8.01 (d, J=7.8 Hz, 1H), 7.40 (d, J=7.8 Hz, 1H), 6.40 (t, J=8.7 Hz, 1H), 5.65 (m, 1H), 4.58-4.50 (m, 3H), 4.23-4.19 (m, 3H), 2.72-2.62 (m, 1H), 2.44-2.38 (m, 1H), 1.70 (m, 1H), 1.42-1.35 (m, 4H), 1.22-1.13 (m, 12H), 0.96 (m, 3H).

### Example 8: Synthesis of [(2R,3R,5R)-3-[[(2S)-2-amino-3-methylbutanoyl]oxy]-4,4-difluoro-5-(2-oxo-4-[[(pentyloxy)carbonyl]amino]-1,2-dihydropyrimidin-1-yl)oxolan-2-yl]methyl (2S)-2-amino-3-methylbutanoate

Into a 100-mL round-bottom flask, was placed pentyl-N-[1-[(2R,4R,5R)-3,3-difluoro-4-hydroxy-5-(hydroxymethyl)oxolan-2-yl]-2-oxo-1,2-dihydropyrimidin-4-yl]carbamate (500 mg, 1.33 mmol, 1.00 equiv), (2S)-2-[[(tert-butoxy)carbonyl]amino]-3-methylbutanoic acid (1.15 g, 5.29 mmol, 4.00 equiv), DCC (1.64 g, 7.96 mmol, 6.00 equiv), 4-dimethylaminopyridine (485 mg, 3.98 mmol, 3.00 equiv), N,N-dimethylformamide (30 mL). The resulting solution was stirred for 2 h at room temperature. The reaction was then quenched by 100 ml of water. The resulting solution was extracted with 100 ml of ethyl acetate and the organic layers combined. The resulting mixture was washed with 2×100 mL of brine. The mixture was dried over anhydrous sodium sulfate and concentrated under vacuum. The crude product was purified by Prep-HPLC. This resulted in 400 mg (39%) of [(2R,3R,5R)-3-[[(2S)-2-[[(tert-butoxy)carbonyl]amino]-3-methylbutanoyl]oxy]-4,4-difluoro-5-(2-oxo-4-[[(pentyloxy)carbonyl]amino]-1,2-dihydropyrimidin-1-yl)oxolan-2-yl]methyl (2R)-2-[[(tert-butoxy)carbonyl]amino]-3-methylbutanoate as a white solid. ¹H-NMR: (300 MHz, d₆-DMSO, ppm): δ 10.87 (br, 1H), 8.06 (d, J=7.8 Hz, 1H), 7.43 (d, J=6.6 Hz, 1H), 7.27 (d, J=7.8 Hz, 1H), 7.27 (d, J=7.8 Hz, 1H), 6.36 (t, J=8.7 Hz, 1H), 5.6-5.42 (br, 1H), 4.51-4.28 (m, 3H), 4.13 (t, J=6.8 Hz, 2H), 3.95 (m, 2H), 2.05 (m, 2H), 1.64 (m, 2H), 1.45-1.29 (m, 22H), 0.96 (m, 15H).

Into a 25-mL round-bottom flask, was placed [(2R,3R,5R)-3-[[(2S)-2-[[(tert-butoxy)carbonyl]amino]-3-methylbutanoyl]oxy]-4,4-difluoro-5-(2-oxo-4-[[(pentyloxy)carbonyl]amino]-1,2-dihydropyrimidin-1-yl)oxolan-2-yl]methyl (2R)-2-[[(tert-butoxy)carbonyl]amino]-3-methylbutanoate (100 mg, 0.13 mmol, 1.00 equiv), hydrogen chloride/Dioxane (8 mL). The resulting solution was stirred for 30 min at room temperature. The resulting mixture was concentrated under vacuum. This resulted in 54 mg (65%) of [(2R,3R,5R)-3-[[(2S)-2-amino-3-methylbutanoyl]oxy]-4,4-difluoro-5-(2-oxo-4-[[(pentyloxy)carbonyl]amino]-1,2-dihydropyrimidin-1-yl)oxolan-2-yl]methyl (2S)-2-amino-3-methylbutanoate as a light brown solid. LC-MS: (M+H)⁺=576. ¹H-NMR: (300 MHz, CD₃OD, ppm): δ8.11 (d, J=7.8 Hz, 1H), 7.27 (d, J=7.8 Hz, 1H), 6.30 (t, J=8.7 Hz, 1H), 5.85 (m, 1H), 4.85-4.65 (m, 3H), 4.27-4.19 (m, 3H), 4.10 (m, 1H), 3.8-3.6 (m, 1H), 2.50-2.32 (m, 2H), 1.76-1.71 (m, 2H), 1.44-1.32 (m, 4H), 1.22-1.06 (m, 12H), 0.96 (m, 3H).

### Biological Example 1: Mice PK Study

The pharmacokinetics of compounds were evaluated in male CD1 mouse via Intravenous and Oral Administration. The iv dose was administered as a slow bolus in the Jugular vein, and oral doses were administered by gavage. The formulation is 2.5% DMSO, 10% EtOH, 20% Cremphor EL, 67.5% D5W. The PK time point 5 min, 15, 30 min, 1, 2, 4, 6, 8 hours post dose. Approximately 0.03 mL blood will be collected at each time point. Keep blood at room temperature and collect plasma within 15 min by centrifugation at 4000 g for 5 minutes in a 4° C. centrifuge. Plasma samples will be stored in polypropylene tubes. The plasma samples will be stored in a freezer at −75±15° C. prior to analysis. Concentrations of compounds and the active metaboliste Gemcitabine in the plasma samples will be analyzed using a LC-MS/MS method. WinNonlin (Phoenix, version 6.1) or other similar software will be used for pharmacokinetic calculations. The following pharmacokinetic parameters will be calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data will be described using descriptive statistics such as mean, standard deviation. Additional pharmacokinetic or statistical analysis may be performed at the discretion of the contributing scientist, and will be documented in the data summary.

The results of oral dosing of 10 mg/kg, as shown in the Table below, show that the Example 2, a novel Triple Prodrug, has better oral exposure of active metabolite Gemcitabine than that of LY2334737. In addition, during the formulation for this PK study, Example 2 shows significant higher water solubility than LY2334737.

| | Example 2 | LY2334737 |
|---|---|---|
| | 10 mg/kg, | 10 mg/kg, |
| | oral dosing | oral dosing |
| Cₘₐₓ (ng/mL) of active | 378 | 127 |
| metabolite Gemcitabine | | |
| AUCₗₐₛₜ (h*ng/mL) of active | 778 | 522 |
| metabolite Gemcitabine | | |

The Table below shows the concentration of the active metabolite Gemcitabine after the single dose of Example 2 in the mice. The result shows good PK linearity and the Cmax of Gemcitabine in the 300 mg/kg is as high as 20,165 nM.

| | | |
|---|---|---|
| | Gemcitabine C_{Max} | Gemcitabine AUCₗₐₛₜ |
| Example 2 | (nM) | (h*ng/mL) |

| | | | |
|---|---|---|---|
| 10 | mg/kg | 1,435 | 778 |
| 150 | mg/kg | 11,698 | 8,220 |
| 300 | mg/kg | 20,165 | 14,783 |

The mice PK studies above confirm that Example 2, is a prodrugs of Gemcitabine, with excellent water solubility.

### Biological Example 2: In Vivo Xenograft Studies

Compound of Example 2 was selected for in vivo studies in the ovarian cancer A2780 xenograft model. Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

Ovarian Cancer is the 5th most common cancer in women: ˜ 22,280 new cases and 14,240 death in 2016 in US. In China, Ovarian Cancer has more than 100,000 new cases each year. Gemcitabine (intravenous dosing) is the 2^{nd} line SOC of Ovarian Cancer. As shown below, Example 2 (oral dosing) has better efficacy than Gemcitabine (IV dosing) in the A2780 model.

| | | | | | | Tumor | |
|---|---|---|---|---|---|---|---|
| Group | mice | Agent | mg/kg | Route | Schedule | volume | |
| 1 | 5 | vehicle | Vehicle | po | q4d × 7 | 2710 | mm³ |
| 2 | 5 | Gemcitabine | 120 | IV | qw × 4 | 442 | mm³ |
| 3 | 5 | Example 2 | 75 | po | q4d × 7 | 65 | mm³ |

What is claimed is:

1. A compound of Formula (I), or an N-oxide thereof, or a pharmaceutically acceptable salt, solvate, polymorph, tautomer, stereoisomer, an isotopic form, or a prodrug of said compound of Formula (I) or N-oxide thereof: 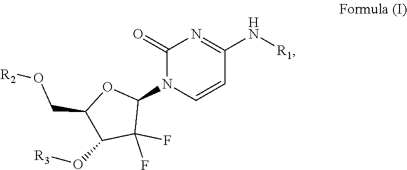
wherein
R₁ is 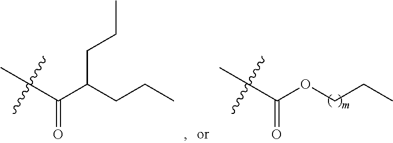
in which m is an integer from 1 to 20; and
each of R₂, and R₃, independently, is

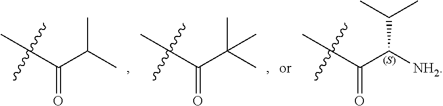

2. A compound according to claim 1 or an N-oxide thereof, or a pharmaceutically acceptable salt, solvate, polymorph or tautomer thereof, wherein the compound is
(2R,3R,5R)-4,4-difluoro-2-((isobutyryloxy)methyl)-5-(2-oxo-4-(2-propylpentanamido)pyrimidin-1(2H)-yl)tetrahydrofuran-3-yl isobutyrate,
(2R,3R,5R)-4,4-difluoro-2-((isobutyryloxy)methyl)-5-(2-oxo-4-(2-propylpentanamido)pyrimidin-1(2H)-yl)tetrahydrofuran-3-yl L-valinate,
((2R,3R,5R)-3-((L-valyl)oxy)-4,4-difluoro-5-(2-oxo-4-(2-propylpentanamido)pyrimidin-1(2H)-yl)tetrahydrofuran-2-yl)methyl valinate,
((2R,3R,5R)-4,4-difluoro-3-(isobutyryloxy)-5-(2-oxo-4-(2-propylpentanamido)pyrimidin-1(2H)-yl)tetrahydrofuran-2-yl)methyl L-valinate,
(2R,3R,5R)-4,4-difluoro-2-((isobutyryloxy)methyl)-5-(2-oxo-4-(((pentyloxy)carbonyl)amino)pyrimidin-1(2H)-yl)tetrahydrofuran-3-yl L-valinate,
((2R,3R,5R)-4,4-difluoro-3-(isobutyryloxy)-5-(2-oxo-4-(((pentyloxy)carbonyl)amino)pyrimidin-1(2H)-yl)tetrahydrofuran-2-yl)methyl L-valinate,
(2R,3R,5R)-4,4-difluoro-2-((isobutyryloxy)methyl)-5-(2-oxo-4-(((pentyloxy)carbonyl)amino)pyrimidin-1(2H)-yl)tetrahydrofuran-3-yl isobutyrate,
((2R,3R,5R)-3-((L-valyl)oxy)-4,4-difluoro-5-(2-oxo-4-(((pentyloxy)carbonyl)amino)pyrimidin-1(2H)-yl)tetrahydrofuran-2-yl)methyl L-valinate,
(2R,3R,5R)-4,4-difluoro-5-(4-(((hexyloxy)carbonyl)amino)-2-oxopyrimidin-1(2H)-yl)-2-((isobutyryloxy)methyl)tetrahydrofuran-3-yl L-valinate,
(2R,3R,5R)-5-(4-((butoxycarbonyl)amino)-2-oxopyrimidin-1(2H)-yl)-4,4-difluoro-2-((isobutyryloxy)methyl)tetrahydrofuran-3-yl L-valinate,
(2R,3R,5R)-4,4-difluoro-5-(2-oxo-4-(2-propylpentanamido)pyrimidin-1(2H)-yl)-2-((pivaloyloxy)methyl)tetrahydrofuran-3-yl L-valinate,
(2R,3R,5R)-4,4-difluoro-5-(4-(((hexyloxy)carbonyl)amino)-2-oxopyrimidin-1(2H)-yl)-2-((pivaloyloxy)methyl)tetrahydrofuran-3-yl L-valinate.

3. A pharmaceutical composition comprising a compound of Formula (I) or an N-oxide thereof as defined in claim 1, or a pharmaceutically acceptable salt, solvate, polymorph, tautomer, stereoisomer, an isotopic form, or a prodrug of said compound of Formula (I) or an N-oxide thereof, and a pharmaceutically acceptable diluent or carrier.

4. A method of treating a neoplastic disease comprising administering to a subject in need thereof an effective amount of a compound of Formula (I) or an N-oxide thereof as defined in claim 1, or a pharmaceutically acceptable salt, solvate, polymorph, tautomer, stereoisomer, an isotopic form, or a prodrug of said compound of Formula (I) or an N-oxide thereof.
